(19) United States
(12) Patent Application Publication
(10) Pub. No.: US 2011/0319369 A1
(43) Pub. Date: Dec. 29, 2011

(54) COMBINATION OF A 17 ALPHA-HYDROXYLASE/C17, 20-LYASE INHIBITOR WITH AN ADDITIONAL THERAPEUTIC AGENT

(75) Inventors: David Casebier, Carlisle, MA (US); Scott Chappel, Milton, MA (US)

(73) Assignee: TOKAI PHARMACEUTICALS, INC., Cambridge, MA (US)

(21) Appl. No.: 13/146,001

(22) PCT Filed: Feb. 5, 2010
(86) PCT No.: PCT/US10/23381

§ 371 Date: Sep. 6, 2011

(30) Foreign Application Priority Data
Feb. 5, 2009 (US) 61/150024

(51) Int. Cl. A61K 31/58; A61P 35/00

(52) U.S. Cl. 514/170; 514/171

(57) ABSTRACT

The invention describes methods of treating cancer in which a therapeutically-effective amount of a 17α-hydroxylase/C_{17,20}-lyase inhibitor is administered to a subject in need thereof, including a subject with a refractory cancer and/or a subject currently undergoing another cancer treatment, wherein the 17α-hydroxylase/C_{17,20}-lyase inhibitor is administered in combination with a therapeutically-effective amount of at least one additional therapeutic agent, including, but not limited to, another anti-cancer agent or a steroid.

### CROSS-REFERENCE

This application is the National Stage of International Application No. PCT/US2010/023381, filed Feb. 5, 2010, which claims the benefit under 35 U.S.C. Sec. 119(e) of U.S. Provisional Application No. 61/150,024, filed Feb. 5, 2009, and which is incorporated herein by reference in its entirety.

### FIELD OF THE INVENTION

The present invention is directed to methods and compositions for treating cancer.

### BACKGROUND OF THE INVENTION

Cancer diagnoses continue to increase, with cancers that are hormone-dependent, namely androgen-related prostate cancer in men and estrogen-related breast cancer in women, meriting special note.

Prostate cancer is currently the second leading cause of cancer-related deaths in men after lung cancer, and second in prevalence only to skin cancer. The primary course of treatment for patients diagnosed with organ-confined prostate cancer is usually prostatectomy or radiotherapy. Not only are these treatments highly invasive and have undesirable side effects, such localized treatments are ineffective on metastatic prostate cancer, and a large percent of individuals who receive these localized treatments will suffer from recurring cancer that is non-localized and resistant to hormone therapy.

In the United States, breast cancer incidence in women has increased from one out of every twenty women in 1960 to one out of every eight women in 2005, and it is the most common cancer among white and African-American women. Most options for women diagnosed with breast cancer, i.e., surgery, radiation and chemotherapy, are also highly invasive and have significant side effects.

Hormone therapy is another treatment option for individuals diagnosed with prostate or breast cancer. Hormone therapy is a form of systemic treatment for prostate or breast cancer, wherein hormone ablation agents are used to suppress the production or block the effects of hormones, such as estrogen and progesterone, which are believed to promote the growth of breast cancer, as well as testosterone and dihydrotestosterone, which are believed to promote the growth of prostate cancer. This therapy is less invasive than surgery and does not have many of the side effects associated with chemotherapy or radiation. In addition, hormone therapy may be used by itself or in addition to localized therapy, and has been shown to be effective in individuals with metastatic neoplasia.

While hormone therapy is less invasive and may be used on more advanced stages of cancer, some individuals administered current hormone therapy treatments may not respond completely, or even partially, to such treatments. Current hormone therapy treatments may offer temporal remission of cancer, but these treated cancers can relapse or recur, and upon recurrence, these cancers often have developed a resistance to hormonal therapy. Due to the typically aggressive nature of these recurrent cancers, and their resistance to hormonal therapy, patients with these conditions are often left with few options for treatment.

Despite the progress made in the treatment of cancer, there remains a need for more effective ways to treat cancer, such as, but not limited to, prostate cancer and breast cancer. Additionally, there is a need for effective anti-cancer treatment options for patients who are not responding to current anti-cancer treatments, as well as for effective anti-cancer treatment options for patients whose cancers have recurred.

### SUMMARY OF THE INVENTION

In some embodiments, the invention provides a method for the treatment of a cancer in a subject, the method comprising administering a therapeutically-effective amount of at least one compound of Formula I:

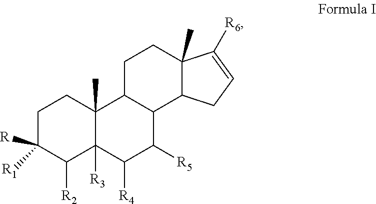

and a therapeutically-effective amount of at least one additional therapeutic agent to a subject having a cancer, wherein:

either R and R₁ are independently H, OH, SH, NH₂, N(R₇), NHR₇, F, OR₇, or O(C═O)R₇; or R and R₁ together form a ketone or an exo-methylene;

each occurrence of R₇ is independently H, C₁-C₈-alkyl, arakyl, alkylaryl, alkoxyalkyl, aryl,

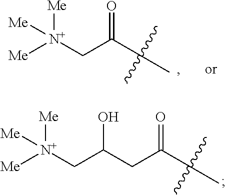

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

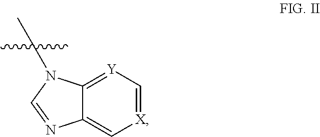

wherein X and Y are independently CH or N, and the bicycle of Formula II is optionally substituted with halogen, chalcogen or C₁-C₄-alkyl,

wherein R₆ is a bicycle of Formula II wherein one of X and Y is N and the other of X and Y is CH when one or both of R and R₁ are

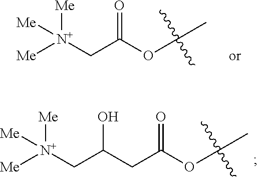

or an analog, a derivative, a metabolite or a pharmaceutically-acceptable salt of any of the foregoing.

In some embodiments, the invention provides a method for treating a subject having a refractory prostate or breast cancer, wherein the subject is receiving at least one other treatment for cancer, the method comprising administering a therapeutically-effective amount of at least one 17α-hydroxylase/C_{17,20}-lyase inhibitor in addition to the other treatment the subject is receiving, wherein the 17α-hydroxylase/C_{17,20}-lyase inhibitor is a compound of Formula 1:

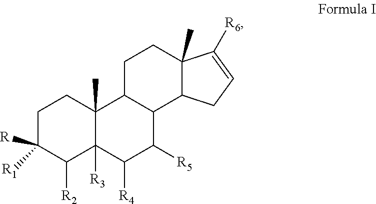

wherein:

either R and R₁ are independently H, OH, SH, NH₂, N(R), NHR₇, F, OR₇, or O(C═O)R₇; or R and R₁ together form a ketone or an exo-methylene;

each occurrence of R₇ is independently H, C₁-C₈-alkyl, arakyl, alkylaryl, alkoxyalkyl, aryl,

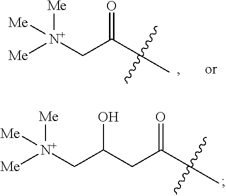

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

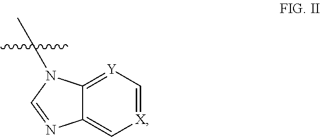

wherein X and Y are independently CH or N, and the bicycle of Formula II is optionally substituted with halogen, chalcogen or C₁-C₄-alkyl,

wherein R₆ is a bicycle of Formula II wherein one of X and Y is N and the other of X and Y is CH when one or both of R and R₁ are

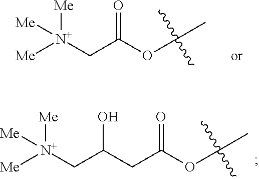

or an analog, a derivative, a metabolite or a pharmaceutically-acceptable salt of any of the foregoing.

In some embodiments, the invention provides a pharmaceutical composition for the treatment of a cancer in a subject, the composition comprising a therapeutically-effective amount of at least one 17α-hydroxylase/C_{17,20}-lyase inhibitor, and at least one additional therapeutic agent, wherein the 17α-hydroxylase/C_{17,20}-lyase inhibitor comprises a compound of Formula (1):

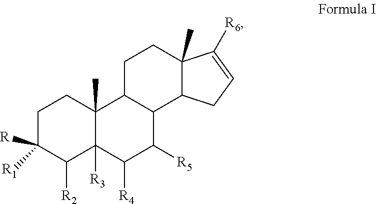

wherein:

R is H or an ester;

R₁ is H, OH, SH, NH₂, N(R₇), NHR₇, F, OR₇, or O(C═O)R₇;

R₇ is independently at each occurrence H, C₁-C₈-alkyl, arakyl, alkylaryl, alkoxyalkyl, or aryl;

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

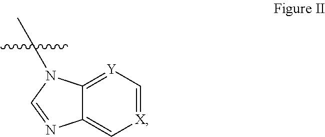

wherein X and Y are independently CH or N, and the bicycle of Formula II is optionally substituted with halogen, chalcogen or C₁-C₄-alkyl,
wherein R₆ is a bicycle of Formula II wherein one of X and Y is N and the other of X and Y is CH when one or both of R and R₁ are

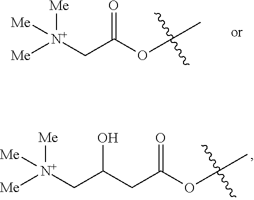

or an analog, a derivative, a metabolite or a pharmaceutically-acceptable salt of any of the foregoing.

In some embodiments, the invention provides a pharmaceutical composition for the treatment of a cancer in a subject comprising a therapeutically-effective amount of Compound I, Compound II, or Compound III, and a therapeutically-effective amount of a steroid, wherein the composition is suitable for oral administration.

### DETAILED DESCRIPTION OF INVENTION

The method described herein for treating cancer comprises administering to a subject, such as a human, a 17α-hydroxylase/C_{17,20}-lyase inhibitor in addition to at least one other therapeutic agent. In some embodiments, the other therapeutic agent is an anti-resorptive agent, a monoclonal antibody, a hormonal ablation agent, an adhesion molecule, a growth factor inhibitor, a proapoptotic agent, an antisense agent, a vitamin D analog, an RNAi agent, a modified peptide, or an enzyme inhibitor. In some embodiments, the other therapeutic agent is an anti-cancer agent, a steroid, or a glucocorticoid. The compositions described herein comprise a 17α-hydroxylase/C_{17,20}-lyase inhibitor and at least one additional therapeutic agent, such as another anti-cancer agent or a steroid, a corticosteroid or a glucocorticoid. In some embodiments, other anti-cancer treatments, such as administration of one or more other anti-cancer agents, radiotherapy, chemotherapy, photodynamic therapy, surgery or other immunotherapy, are used with the methods and compositions of the invention.

As used herein, and unless otherwise defined, the following terms have the meanings provided:

“Cancer” refers to the growth, division or proliferation of abnormal cells in the body. Cancers that can be treated with the methods and the compositions described herein include, but are not limited to, prostate cancer, breast cancer, adrenal cancer, leukemia, lymphoma, myeloma, Waldenstrom's macroglobulinemia, monoclonal gammopathy, benign monoclonal gammopathy, heavy chain disease, bone and connective tissue sarcoma, brain tumors, thyroid cancer, pancreatic cancer, pituitary cancer, eye cancer, vaginal cancer, vulvar cancer, cervical cancer, uterine cancer, ovarian cancer, esophageal cancer, stomach cancer, colon cancer, rectal cancer, liver cancer, gallbladder cancer, cholangiocarcinoma, lung cancer, testicular cancer, penile cancer, oral cancer, skin cancer, kidney cancer, Wilms' tumor and bladder cancer.

“Recurring cancer” means cancer that has returned after a patient has been earlier diagnosed with cancer, has undergone treatment and/or had been previously diagnosed as cancer-free.

“Relapse cancer” means cancer that was at one time responsive to an anti-cancer treatment, but has become no longer responsive to such treatment or is no longer responding sufficiently to such treatment.

“Refractory cancer” means a cancer that is not responding to an anti-cancer treatment or cancer that is not responding sufficiently to an anti-cancer treatment, including recurring or relapse cancer.

“Treat,” “treating” and “treatment” include the eradication, removal, modification, management or control of a tumor or primary, regional, or metastatic cancer cells or tissue and the minimization or delay of the spread of cancer.

“Subject” means an animal, including but not limited to a mammal, such as a human, monkey, cow, horse, sheep, pig, chicken, turkey, quail, cat, dog, mouse, rat, rabbit, or guinea pig. In one embodiment the subject is a mammal and in another embodiment the subject is a human. In some embodiments, the subject is an adult male or an adult female. In some embodiments, the subject is a male of age about 30 years to about 85 years. In some embodiments, the subject is a female of age about 30 years to about 85 years. In some embodiments, the subject has or is susceptible to having cancer. In some embodiments, the subject has or is susceptible to having a tumor. In some embodiments, the subject is castrated. In some embodiments, the subject is non-castrated.

“17α-Hydroxylase/C_{17,20}-lyase inhibitor” or “inhibitor” refers to an inhibitor of 17α-hydroxylase/C_{17,20}-lyase, an analog thereof, derivative thereof, metabolite thereof or pharmaceutically-acceptable salt thereof. Also, unless otherwise noted, reference to a particular 17α-hydroxylase/C_{17,20}-lyase inhibitor can include analogs, derivatives, metabolites or pharmaceutically-acceptable salts of such particular 17α-hydroxylase/C_{17,20}-lyase inhibitor.

“Hormonal agent” includes, but is not limited to, “androgen ablation agents” and “estrogen ablation agents,” and, whether used as “a hormonal ablation agent” or “the hormonal ablation agent,” “hormonal ablation agent” should not be interpreted as being limited to the inclusion of a single hormonal ablation agent.

“Anti-cancer agent” refers to any therapeutic agent that directly or indirectly kills cancer cells or directly or indirectly prohibits, stops or reduces the proliferation of cancer cells. It should be noted that, even though the phrase “anti-cancer agent” may be written as a singular noun, for example, “an anti-cancer agent” or “the anti-cancer agent,” the phrase “anti-cancer agent” should be interpreted as referring to one or more anti-cancer agents.

“Pharmaceutically-acceptable salt”, refers to any pharmaceutical salt suitable for administration to a subject. Non-limiting examples of pharmaceutically-acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, formates, isobutyrates, caproates, heptanoates, propiolates, oxalates, malonates, succinates, suberates, sebacates, fumarates, maleates, butyne-1,4-dioates, hexyne-1,6-dioates, malates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartrates, alkanesulfonates (e.g., methane-sulfonate or mesylate), trifluoromethylsulfonates, propanesulfonates, naphthalene-1-sulfonates, naphthalene-2-sulfonates, and mandelates. Several of the pharmaceutically-approved salts are listed in Remington: The Science and Practice of Pharmacy, Mack Publishing Co., Easton, Pa.

The following terms have the following meanings, unless otherwise specified. Alkyl is a C₁-C₁₂-straight, C₁-C₁₂-branched, or C₁-C₁₂-cyclic carbogenic group, which is optionally substituted independently at each position. Non-limiting examples of substituents include hydroxyl, methoxy, ethoxy, sulfhydryl, methyl mercapto, ethylmercapto, fluorine, chlorine, bromine, iodine, aryl, and heteroaryl.

Aryl is a mono- or polycyclic aromatic ring system, which is optionally substituted independently at each position. Non-limiting examples of aryl include phenyl, naphthyl, indenyl, fluorenyl, phenathrenyl, azulenyl, or C₅-C₁₀ aromatic groups. Non-limiting examples of substituents include hydroxyl, methoxy, ethoxy, sulfhydryl, methylmercapto, ethylmercapto, fluorine, chlorine, bromine, iodine, oxo, or heteroaryl.

Heteroaryl is a mono- or polycyclic aromatic system, which is optionally substituted independently at each position, containing at least one ring heteroatom selected from nitrogen, oxygen, and sulfur. Non-limiting examples of substituents include hydroxyl, methoxy, ethoxy, sulfhydryl, methyl mercapto, ethylmercapto, fluorine, chlorine, bromine, iodine, oxo and aryl. Non-limiting examples of heteroaryl include furan, thiophene, pyrrole, pyrrazole, imidazole, oxazole, isoxazole, thiazole, isothiazole, triazole, thiadiazole, oxadiazole, pyridine, pyrimidine, pyrazine, pyridazine, triazine, indole, carbazole, benzofuran, benzothiphene, benzthiazole, indazole, quinoline, isoquinoline, cinnoline, phthalazine, and heteroaryl groups containing from five-to-twelve or ring atoms.

Aralkyl refers to an aryl group that is distally attached via an alkyl group, for example, benzyl.

Alkylaryl refers to an alkyl group that is distally attached via an aryl group, for example, o-, m-, or p-toluoyl.

Polyalkoxyl is poly(propylene glycol) or poly(ethylene glycol), wherein the monomers are repeated 2-100 times, where such polyalkoxy groups may be defined by the precise range of repeating units (e.g., 35-40), by the targeted peak of envelope distribution in the repeating units (e.g., 114 from PEG5000), or by a selection for solubility or physical properties. Polyalkoxyl groups may also be “capped” by an alkyl group (MPEG5000 for methoxy-PEG5000) or an aryl group, such as phenyl (polyalkoxylaryl).

17α-hydroxylase/C_{17,20}-lyase inhibitors

17α-hydroxylase/C_{17,20}-lyase inhibitors have been shown to be useful in the treatment of cancer, specifically hormone-dependent disorders, such as androgen-dependent and estrogen-dependent disorders like prostate cancer and breast cancer, respectively, as described in U.S. Pat. No. 5,604,213 (Barrie et al.), which is incorporated in its entirety herein by reference.

In some embodiments, the 17α-hydroxylase/C_{17,20}-lyase inhibitor is 17-(1-benzimidazolyl)androsta-5,16-dien-3β-ol; 17-(1-benzimidazolyl)androsta-3,5,16-triene; 17-(1-benzimidazolyl)androsta-4,16-dien-3-one; 17-(1-benzimidazolyl)estra-1,3,5[10],16-tetraen-3-ol; 17-(1-benzimidazolyl)-5α-androst-16-en-3α-ol; 17-(1-benzimidazolyl)-5α-androst-16-en-3-one; 17-(1-benzimidazolyl)androsta-4,16-diene-3,11-dione; 17-(1-benzimidazolyl)-androsta-3,5,16-trien-3-ol; 6□-fluoro-17-(1-benzimidazolyl)androsta-4,16-dien-3-one; 6β-fluoro-17-(1-benzimidazolyl)androsta-4,16-dien-3-one; 17-(1-benzimidazolyl)androsta-4,16-dien-3,6-dione; 3α-trifluoromethyl-17-(1-benzimidazolyl)-androst-16-en-3β-ol, or an acid addition salt, ester, metabolite, analog, derivative or pharmaceutically-acceptable salt thereof. In some embodiments, the 17α-hydroxylase/C_{17,20}-lyase inhibitor is 17-(1-azaazulen-3-yl)androsta-5,16-dien-3β-ol; 17-(2-alkylindazol-3-yl)androsta-5,16-dien-3α-ol; 17-(pyrazolo-[1,5-a]-pyridin-3-yl)androsta-5,16-dien-3β-ol; 17-(imidazo-[1,2-a]-pyridin-3-yl)androsta-5,16-dien-3β-ol; 17-(pyrazolo-[2,3-a]-pyrimidin-3-yl)androsta-5,16-dien-3β-ol; 17-(pyrazolo-[2,3-c]-pyrimidin-3-yl)androsta-5,16-dien-3β-ol; 17-(imidazo-[1,2-c]-pyrimidin-3-yl)androsta-5,16-dien-3β-ol; 17-(imidazo-[1,2-a]-pyrimidin-3-yl)androsta-5,16-dien-3β-ol; 17-(4-alkylpyrazolo-[1,5-a]imidazol-3-yl)androsta-5,16-dien-3β-ol; 17-(2,1-benzoxazol-3-yl)androsta-5,16-dien-3α-ol; 17-(2,1-benzthiazol-3-yl)androsta-5,16-dien-3β-ol; 17-(imidazo[2,1-b][1,3]oxazol-5-yl)androsta-5,16-dien-3β-ol; 17-(imidazo[2,1-b][1,3]thiazol-5-yl)androsta-5,16-dien-3β-ol; or 17-(imidazo-[2,1-b][1,2]isoxazol-6-yl)androsta-5,16-dien-3β-ol; or an acid addition salt, ester, metabolite, analog, derivative or pharmaceutically-acceptable salt thereof.

In some embodiments, the 17α-hydroxylase/C_{17,20}-lyase inhibitor is a compound of the Formula (1):

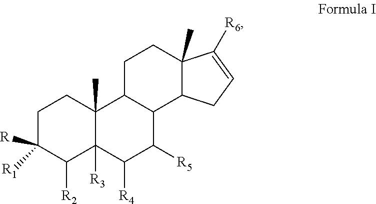

wherein:
either R and R₁ are independently H, OH, SH, NH₂, N(R₇), NHR₇, F, OR₇, or

O(C═O)R₇; or R and R₁ together form a ketone or an exo-methylene;

each occurrence of R₇ is independently H, C₁-C₈-alkyl, arakyl, alkylaryl, alkoxyalkyl, aryl,

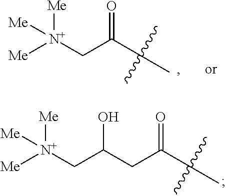

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

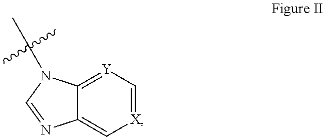

wherein X and Y are independently CH or N, and the bicycle of Formula II is optionally substituted with halogen, chalcogen or C₁-C₄-alkyl,

wherein R₆ is a bicycle of Formula II wherein one of X and Y is N and the other of X and Y is CH when one or both of R and R₁ are

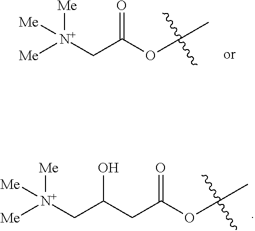

In some embodiments, R₆ is 9-purinyl, 7-purinyl, 7-(6-amino)purinyl, 1-benzimidazolyl, and 1-benztriazolyl.

In some embodiments, R is hydroxyl or an ester.

In some embodiments, the 17α-hydroxylase/C_{17,20}-lyase inhibitor is Compound I, having the structural formula:

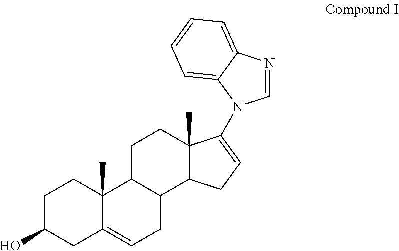

or a pharmaceutically-acceptable salt thereof.

In some embodiments, the 17α-hydroxylase/C_{17,20}-lyase inhibitor is Compound II, having the structural formula:

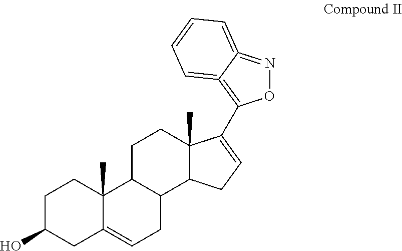

or a pharmaceutically-acceptable salt thereof.

In some embodiments, the 17α-hydroxylase/C_{17,20}-lyase inhibitor is Compound III, having the structural formula:

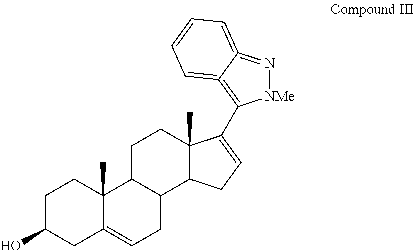

or a pharmaceutically-acceptable salt thereof.

The 17α-hydroxylase/C_{17,20}-lyase inhibitor may be made according to any method known to one skilled in the art. For example, such inhibitors may be synthesized according to the method disclosed in U.S. Pat. Nos. 5,994,335 and 6,444,683 (both Brodie et al), both herein incorporated by reference each in its entirety. Another method of making 17α-hydroxylase/C_{17,20}-lyase inhibitors is disclosed in U.S. patent application Ser. No. 09/749,871 to Brodie and Njar, herein also incorporated by reference in its entirety.

The amount of 17α-hydroxylase/C_{17,20}-lyase inhibitor administered to a subject having cancer is an amount that is sufficient to treat the cancer. In some embodiments, the 17α-hydroxylase/C_{17,20}-lyase inhibitor is administered alone. In some embodiments, the 17α-hydroxylase/C_{17,20}-lyase inhibitor is administered in combination with an additional anti-cancer treatment, such as an additional anti-cancer agent.

### Additional Therapeutic Agents

Suitable compounds that may be used, in addition to 17α-hydroxylase/C_{17,20}-lyase inhibitors, as anti-cancer agents include, but are not limited to, hormone ablation agents, anti-androgen agents/anti-androgens, differentiating agents, anti-neoplastic agents, kinase inhibitors, anti-metabolite agents, alkylating agents, antibiotic agents, immunological agents, interferon-type agents, intercalating agents, growth factor inhibitors, cell-cycle inhibitors, enzymes, topoisomerase inhibitors, biological response modifiers, mitotic inhibitors, matrix metalloprotease inhibitors, anti-resorptives, monoclonal antibodies, adhesion molecules, growth factors, proapoptotic agents, antisense agents, vitamin D analogs, RNAi agents, modified peptides, enzyme inhibitors, agents ameliorating the side effects of therapy, and genetic therapeutics. In some embodiments, the amount of the additional anti-cancer agent administered to a subject having cancer is an amount that is sufficient to treat the cancer. Examples of some of the above classes of anti-cancer agents are listed below for purposes of illustration and not for purposes of limitation, as these examples are not all-inclusive. Many of the examples below could be listed in multiple classes of anti-cancer agents and are not restricted in any way to the class in which they are listed.

In some embodiments, the 17α-hydroxylase/C_{17,20}-lyase inhibitor is administered with a hormonal ablation agent, such as deslorelin, leuprolide, goserelin or triptorelin. In some embodiments, the amount of the hormonal ablation agent administered to a subject having cancer is an amount that is sufficient to treat the cancer.

Non-limiting examples of anti-androgen agents include bicalutamide, flutamide, spironolactone, cyproterone acetate, finasteride, dutasteride, and nilutamide. In some embodiments, the amount of the anti-androgen agent administered to a subject having cancer is an amount that is sufficient to treat the cancer. In some embodiments the 17α-hydroxylase/C_{17,20}-lyase inhibitor is administered with an antiestrogen agent, including but not limited to tamoxifen.

In another embodiment, the 17α-hydroxylase/C_{17,20}-lyase inhibitor is administered with a differentiating agent. Non-limiting examples of differentiating agents include polyamine inhibitors; vitamin D and its analogs, such as calcitriol, doxercalciferol, ergocalciferol, 22-oxacalcitriol, dihydrotachysterol, paricalcitol, and seocalcitol; inhibitors of vitamin A metabolism, such as RAMBAS, e.g. liarozole, metabolites of vitamin A, such as ATRA; retinoic acid; retinoids; short-chain fatty acids; phenylbutyrate; and nonsteroidal anti-inflammatory agents. In some embodiments, the amount of the differentiating agent administered to a subject having cancer is an amount that is sufficient to treat the cancer.

In another embodiment, the 17α-hydroxylase/C_{17,20}-lyase inhibitor is administered with an anti-neoplastic agent. Non-limiting examples of anti-neoplastic agents include tubulin interacting agents, topoisomerase inhibitors and agents, acitretin, alstonine, amonafide, amphethinile, amsacrine, ankinomycin, anti-neoplaston, aphidicolin glycinate, asparaginase, baccharin, batracylin, benfluoron, benzotript, bromofosfamide, caracemide, carmethizole hydrochloride, chlorsulfaquinoxalone, clanfenur, claviridenone, crisnatol, curaderm, cytarabine, cytocytin, dacarbazine, datelliptinium, dihaematoporphyrin ether, dihydrolenperone, dinaline, distamycin, docetaxel, elliprabin, elliptinium acetate, epothilones, ergotamine, etoposide, etretinate, fenretinide, gallium nitrate, genkwadaphnin, hexadecylphosphocholine, HDAC inhibitors, homoharringtonine, hydroxyurea, ilmofosine, isoglutamine, isotretinoin, leukoregulin, lonidamine, merbarone, merocyanlne derivatives, methylanilinoacridine, minactivin, mitonafide, mitoquidone, mitoxantrone, mopidamol, motretinide, N-(retinoyl)amino acids, N-acylated-dehydroalanines, nafazatrom, nocodazole derivative, octreotide, oquizanocine, paclitaxel, pancratistatin, pazelliptine, piroxantrone, polyhaematoporphyrin, polypreic acid, probimane, procarbazine, proglumide, razoxane, retelliptine, spatol, spirocyclopropane derivatives, spirogermanium, strypoldinone, superoxide dismutase, teniposide, thaliblastine, tocotrienol, topotecan, ukrain, vinblastine sulfate, vincristine, vindesine, vinestramide, vinorelbine, vintriptol, vinzolidine, and withanolides. In some embodiments, the amount of the anti-neoplastic agent administered to a subject having cancer is an amount that is sufficient to treat the cancer.

In some embodiments, the 17α-hydroxylase/C_{17,20}-lyase inhibitor is used with a kinase inhibitor. Non-limiting examples of kinase inhibitors include p38 inhibitors; CDK inhibitors; TNF inhibitors; matrixmetallo proteinase (MMP) inhibitors; COX-2 inhibitors, including celecoxib, rofecoxib, parecoxib, valdecoxib, and etoricoxib; SOD mimics; and αᵥβ₃-inhibitors. In some embodiments, the amount of the kinase inhibitor administered to a subject having cancer is an amount that is sufficient to treat the cancer.

In another embodiment, the 17α-hydroxylase/C_{17,20}-lyase inhibitor is administered with an anti-metabolite agent. Non-limiting examples of anti-metabolite agents include 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, inhibitors of essential amino acids, isopropyl pyrrolizine, methobenzaprim, methotrexate, norspermidine, ornithine decarboxylantion inhibitors, pentostatin, piritrexim, plicamycin, thioguanine, tiazofurin, trimetrexate, tyrosine kinase inhibitors, and uricytin. In some embodiments, the amount of the anti-metabolite agent administered to a subject having cancer is an amount that is sufficient to treat the cancer.

In another embodiment, the 17α-hydroxylase/C_{17,20}-lyase inhibitor is administered with an alkylating agent. Non-limiting examples of alkylating agents include aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, elmustine, estramustine phosphate sodium, fotemustine, hepsul-fam, ifosfamide, iproplatin, lomustine, mafosfamide, mitolactol, oxaliplatin, prednimustine, ranimustine, semustine, spiromustine, tauromustine, temozolomide, teroxirone, tetraplatin and trimelamol. In some embodiments, the amount of the alkylating agent administered to a subject having cancer is an amount that is sufficient to treat the cancer, whether administered alone or in combination with a 17α-hydroxylase/C_{17,20}-lyase inhibitor.

In another embodiment, the 17α-hydroxylase/C_{17,20}-lyase inhibitor is administered with an antibiotic agent. Non-limiting examples of antibiotic agents include aclarubicin, actinomycin D, actinoplanone, adriamycin, aeroplysinin derivative, amrubicin, anthracycline, azinomycin-A, bisucaberin, bleomycin sulfate, bryostatin-1, calichemycin, chromoximycin, dactinomycin, daunorubicin, ditrisarubicin B, doxorubicin, doxorubicin-fibrinogen, elsamicin-A, epirubicin, erbstatin, esorubicin, esperamicin-Al, esperamicin-Alb, fostriecin, glidobactin, gregatin-A, grincamycin, herbimycin, idarubicin, illudins, kazusamycin, kesarirhodins, menogaril, mitomycin, neoenactin, oxalysine, oxaunomycin, peplomycin, pilatin, pirarubicin, porothramycin, pyrindanycin A, rapamycin, rhizoxin, rodorubicin, sibanomicin, siwenimycin, sorangicin-A, sparsomycin, talisomycin, terpentecin, thrazine, tricrozarin A, and zorubicin. In some embodiments, the amount of the antibiotic agent administered to a subject having cancer is an amount that is sufficient to treat the cancer.

In some embodiments, the 17α-hydroxylase/C_{17,20}-lyase inhibitor is used with other anti-cancer agents. Non-limiting examples of anti-cancer agents include acemannan, aclarubicin, aldesleukin, alemtuzumab, alitretinoin, altretamine, amifostine, amsacrine, anagrelide, anastrozole, ancestim, bexarotene, broxuridine, capecitabine, celmoleukin, cetrorelix, cladribine, clotrimazole, daclizumab, dexrazoxane, dilazep, docosanol, doxifluridine, bromocriptine, carmustine, cytarabine, diclofenac, edelfosine, edrecolomab, eflornithine, emitefur, exemestane, exisulind, fadrozole, erythropoietin, filgrastim, finasteride, fludarabine phosphate, formestane, fotemustine, gallium nitrate, gemcitabine, glycopine, heptaplatin, ibandronic acid, imiquimod, iobenguane, irinotecan, irsogladine, lanreotide, leflunomide, lenograstim, lentinan sulfate, letrozole, liarozole, lobaplatin, lonidamine, masoprocol, melarsoprol, metoclopramide, mifepristone, miltefosine, mirimostim, mitoguazone, mitolactol, molgramostim, nafarelin, nartograstim, nedaplatin, nilutamide, noscapine, oprelvekin, osaterone, oxaliplatin, pamidronic acid, pegaspargase, pentosan polysulfate sodium, pentostatin, picibanil, pirarubicin, porfimer sodium, raloxifene, raltitrexed, rasburicase, rituximab, romurtide, sargramostim, sizofuran, sobuzoxane, sonermin, suramin, tasonermin, tazarotene, tegafur, temoporfin, temozolomide, teniposide, tetrachlorodecaoxide, thalidomide, thrombopoietin, thymalfasin, thyrotropin alfa, topotecan, toremifene, trastuzumab, treosulfan, tretinoin, trilostane, trimetrexate, ubenimex, valrubicin, verteporfin, and vinorelbine. In some embodiments, the amount of the additional anti-cancer agent(s) administered to a subject having cancer is an amount that is sufficient to treat the cancer.

In some embodiments, the 17α-hydroxylase/C_{17,20}-lyase inhibitor is administered or combined with steroids, corticosteroids, or glucocorticoids. Non-limiting examples steroids include eplerenone, hydrocortisone, prednisone, and dexamethasone. In some embodiments, the 17α-hydroxylase/C_{17,20}-lyase inhibitor and the steroid are administered in the same compositions. In some embodiments, the 17α-hydroxylase/C_{17,20}-lyase inhibitor and the steroid are administered in different compositions. In some embodiments, the amount of the steroid administered to a subject having cancer is sufficient to treat the cancer.

In some embodiments, the 17α-hydroxylase/C_{17,20}-lyase inhibitor is administered with an enzyme inhibitor. In some embodiments, the enzyme inhibitor is an inhibitor of 17α-hydroxylase/C_{17,20}-lyase. In some embodiments, the enzyme inhibitor is an imidazole, and azole or an antifungal agent, for example, ketoconazole.

In some embodiments, the 17α-hydroxylase/C_{17,20}-lyase inhibitor is administered with a monoclonal antibody. In some embodiments, the monoclonal antibody has anti-cancer activity. Non-limiting examples of monoclonal antibodies include rituximab, trastuzumab, gemtuzumab, ozogamicin, alemtuzumab, ibritumomab, tiuxetan, tositumomab, cetuximab, bevacizumab, panitumumab, and ofatumumab.

In some embodiments, the 17α-hydroxylase/C_{17,20}-lyase inhibitor is administered with an antisense agent. In some embodiments, the antisense agent has anti-cancer activity. Non-limiting examples of anti-sense agents include Genasense (oblimersen), Affinitak (ISIS 3521), ISIS 112989 (OGX 011), ISIS 23722, AP 12009, GEM 231, GEM 240, IGF-1R/AS ODN, MG98, LErafAON, Ki-67 antisense oligonucleotide, GTI-2040, ISIS 2503, and AP11014.

In some embodiments, the 17α-hydroxylase/C_{17,20}-lyase inhibitor is administered with a peptide and/or a modified peptide. In some embodiments, the peptide or modified peptide is a ligand. In some embodiments, the peptide or modified peptide is an inhibitor. In some embodiments, the peptide or modified peptide is a vaccine. In some embodiments, the peptide or modified peptide provides a T-cell response against cancer cells. In some embodiments, the peptide or modified peptide has anti-cancer activity.

In some embodiments, the 17α-hydroxylase/C_{17,20}-lyase inhibitor is administered with a RNAi therapy. In some embodiments, the RNAi therapy has anti-cancer activity.

In some embodiments, the 17α-hydroxylase/C_{17,20}-lyase inhibitor is administered with an agent ameliorating the side effects of therapy. In some embodiments, the agent ameliorating the side effects of therapy is a diuretic, a pain reliever, an analgesic, an anti-inflammatory agent, eplerenone, prednisone, proton pump inhibitors, H2 receptor antagonists, lipid lowering agents, antiresorptive agents, antipsychotic agents, or dexamethasone.

In one embodiment, the invention provides methods and compositions comprising both inhibitors and a steroid, a corticosteroid, or a glucocorticoid. Non-limiting examples of steroids include: (1) hydrocortisone (cortisol; cyprionate oral; sodium phosphate injection; sodium succinate; cortisone acetate oral or injection forms, etc.), (2) dexamethasone (e.g., DECADRON® oral; Decadron®-LA injection, etc.), (3) prednisolone (e.g., DELTA-CORTEF® prednisolone sodium succinate, prednisolone acetate, prednisolone sodium phosphate, prednisolone tebutate), or (4) prednisone and combinations thereof. See, e.g., Goodman & Gilman's The Pharmacological Basis of Therapeutics, 10^{th} sup. Edition 2001.

In some embodiments, a solid oral dosage form comprises from about 20 mg to about 500 mg of an inhibitor and from about 0.5 mg to about 3.0 mg of a steroid, e.g., a glucocorticoid, optionally with one or more excipients, carriers, or diluents. In some embodiments, the dosage form comprises about 250 mg of Compound I and about 1.0 mg, 1.25 mg, 1.5 mg, or 2.0 mg of a steroid, for example, a corticosteroid or a glucocorticoid.

Administration of the 17α-hydroxylase/C_{17,20}-lyase inhibitor and an Additional Therapeutic Agent

In some embodiments, the 17α-hydroxylase/C_{17,20}-lyase inhibitor and the additional therapeutic agent are administered by any method known to one skilled in the art. In some embodiments, the 17α-hydroxylase/C_{17,20}-lyase inhibitor and the additional therapeutic agent are in separate compositions prior to administration. In some embodiments, the 17α-hydroxylase/C_{17,20}-lyase inhibitor and the additional therapeutic agent are combined into the same composition for administration.

In some embodiments, the 17α-hydroxylase/C_{17,20}-lyase inhibitor and the additional therapeutic agent are administered sequentially. In some embodiments, the 17α-hydroxylase/C_{17,20}-lyase inhibitor is administered before the additional therapeutic agent. In some embodiments, the 17α-hydroxylase/C_{17,20}-lyase inhibitor is administered after the additional therapeutic agent. In some embodiments, the 17α-hydroxylase/C_{17,20}-lyase inhibitor and the additional therapeutic agent are administered simultaneously.

Non-limiting examples of modes of administration include parenteral (e.g., subcutaneous, intramuscular, intraorbital, intracapsular, intraspinal, intrasternal, intravenous, intradermal, intraperitoneal, intraportal, intra-arterial, intrathecal, transmucosal, intra-articular, and intrapleural), transdermal (e.g., topical), epidural, and mucosal (e.g., intranasal) injection or infusion, as well as oral, inhalation, pulmonary, and rectal administration. In some embodiments, a 17α-hydroxylase/C_{17,20}-lyase inhibitor and an additional therapeutic agent are administered orally.

In some embodiments, the 17α-hydroxylase/C_{17,20}-lyase inhibitor is administered transdermally. In some embodiments, the additional therapeutic agent is administered parenterally. In some embodiments, the 17α-hydroxylase/C_{17,20}-lyase inhibitor is administered orally. Non-limiting examples of oral administration forms include liquid suspensions, tablets, caplets and capsules. In some embodiments, the additional therapeutic agent is administered intravenously. Non-limiting examples of intravenous-administered therapeutic agents include docetaxel injections, such as Taxotere®; paclitaxel injections, such as Paclitaxel®; and mitoxantrone injections, such as Novantrone®. In some embodiments, the additional therapeutic agent is administered in the form of depots or implants. Non-limiting examples of depots or implants include leuprolide depots and implants, e.g., Viadur®. and Lupron Depot®; triptorelin depots, e.g., Trelstar®; goserelin implants, e.g., Zoladex®.

The suitable daily dosage of the 17α-hydroxylase/C_{17,20}-lyase inhibitor depends upon a number of factors, including the nature of the severity of the condition to be treated, the particular inhibitor employed, the route of administration, and the age, weight, and response of the individual subject. In some embodiments, daily dosages of 17α-hydroxylase/C_{17,20}-lyase inhibitors range from about 0.01 to about 1000 mg/kg/day, from about 0.01 to about 100 mg/kg/day, from about 0.1 mg/kg/day to about 1000 mg/kg/day, or from about 1 mg/kg/day to about 200 mg/kg/day, or from about 10 mg/kg/day to about 200 mg/kg/day, or from about 1 mg/kg/day to about 100 mg/kg/day. In some embodiments, the 17α-hydroxylase/C_{17,20}-lyase inhibitor is administered in a single dose. In some embodiments, the 17α-hydroxylase/C_{17,20}-lyase inhibitor is administered in multiple doses.

In some embodiments, the 17α-hydroxylase/C_{17,20}-lyase inhibitor is administered in an amount of greater than about 0.001 mg/day, 0.01 mg/day, 0.1 mg/day, 0.5 mg/day, 1 mg/day, 5 mg/day, 10 mg/day, 25 mg/day, 50 mg/day, 100 mg/day, 250 mg/day, 500 mg/day, or 1000 mg/day. In some embodiments, the 17α-hydroxylase/C_{17,20}-lyase inhibitor is administered in an amount of less than about 5000 mg/day, 4000 mg/day, 3000 mg/day, 2500 mg/day, 2000 mg/day, 1800 mg/day, 1500 mg/day, or 1000 mg/day. In some embodiments, the 17α-hydroxylase/C_{17,20}-lyase inhibitor is administered in an amount from about 0.004 mg/day to about 5000 mg/day, or from about 0.04 mg/day to about 3000 mg/day, or from about 0.4 mg/day to about 1500 mg/day. In some embodiments, the 17α-hydroxylase/C_{17,20}-lyase inhibitor is administered in an amount from about 0.01 mg/day to about 2000 mg/day, or from about 0.1 mg/day to about 2000 mg/day, or from about 1 mg/day to about 2000 mg/day, or from about 10 mg/day to about 2000 mg/day, or from about 20 mg/day to about 2000 mg/day, or from about 50 mg/day to about 2000 mg/day, or from about 100 mg/day to about 1500 mg/day, or from about 5 mg/day to about 1000 mg/day, or from about 5 mg/day to about 900 mg/day, or from about 10 mg/day to about 800 mg/day, or from about 15 mg/day to about 700 mg/day, or from about 20 mg/day to about 600 mg/day, or from about 25 mg/day to about 500 mg/day, or from about 25 mg/day to about 400 mg/day, or from about 25 mg/day to about 300 mg/day, or from about 25 mg/day to about 250 mg/day, or from about 50 mg/day to about 200 mg/day. In some embodiments, the 17α-hydroxylase/C_{17,20}-lyase inhibitor is administered in a single dose. In some embodiments, the 17α-hydroxylase/C_{17,20}-lyase inhibitor is administered in multiple doses. In some embodiments, the 17α-hydroxylase/C_{17,20}-lyase inhibitor is co-administered with an additional anti-cancer agent, such as mitoxantrone, paclitaxel or docetaxel. In some embodiments, the method for the treatment of a cancer in a subject comprises administering about 0.01 mg/kg/day to about 100 mg/kg/day of an inhibitor and about 0.1 mg/m² to about 20 mg/m² of mitoxantrone. In some embodiments, the mitoxantrone is administered over a period of between about 10 to about 20 minutes, once every 21 days.

In some embodiments, a method for the treatment of a cancer in a subject comprises administering about 0.01 mg/kg/day to about 100 mg/kg/day of an inhibitor and about 1 mg/m² to about 175 mg/m² of paclitaxel. In some embodiments, the paclitaxel is administered over a period of between about 2 to about 5 hours, once every three months.

In some embodiments, a method for the treatment of a cancer in a subject comprises administering about 0.01 mg kg/day to about 100 mg/kg/day of an inhibitor and about 1 mg/m² to about 100 mg/m² of docetaxel. In some embodiments, the docetaxel is administered over a period of between about 1 to about 2 hours, once every three weeks.

In some embodiments, the 17α-hydroxylase/C_{17,20}-lyase inhibitor is co-administered with an anti-cancer agent that is a hormonal ablation agent, including, but not limited to, leuprolide, goserelin, triptorelin, a GnRH agonist, or a GnRH antagonist. Non-limiting examples of GnRH agonists include buserelin, deslorelin, leuprolide, goserelin, nafarelin, and histrelin. Non-limiting examples of GnRH antagonists include abarelix, degarelix, ganirelix, cetrorelix. In some embodiments, a method for the treatment of a cancer in a subject comprises administering about 0.01 mg/kg/day to about 100 mg/kg/day of an inhibitor and about 0.01 mg to about 200 mg of leuprolide over a period of about 3 days to about 12 months. In some embodiments, the leuprolide is administered in the amount of about 3.6 mg of leuprolide over a period of about 3 days to about 12 months.

In some embodiments, a method for the treatment of cancer in a subject include administering 0.01 mg/kg/day to about 100 mg/kg/day of an inhibitor and about 0.01 mg to about 20 mg of goserelin over a period of about 28 days to about 3 months. In some embodiments, the goserelin is administered in the amount of about 3.6 mg to about 10.8 mg over a period of about 28 days to about 3 months.

In certain embodiments, the methods for the treatment of cancer in a subject comprises administering about 0.01 mg/kg/day to about 100 mg/kg/day of an inhibitor and about 0.01 mg to about 20 mg of triptore linover a period of about 1 month. In some embodiments, the triptorelin is administered in the amount of about 3.75 mg over a period of about 1 month.

In one embodiment, the method for the treatment of a cancer in a subject comprises administering about 0.01 mg/kg/day to about 100 mg/kg/day of an inhibitor and 0.1 μg/day to about 500 μg/day of seocalcitol. In some embodiments, about 100 μg/day of seocalcitol is administered.

In another embodiment, the method for the treatment of a cancer in a subject comprises administering about 0.01 mg/kg/day to about 100 mg/kg/day of an inhibitor and about 1 mg/day to about 300 mg/day of bicalutamide.

In another embodiment, the method for the treatment of a cancer in a subject comprises administering 0.01 mg/kg/day to about 100 mg/kg/day of an inhibitor and about 1 mg/day to about 2000 mg/day of flutamide.

In some embodiments, the method for the treatment of a cancer in a subject comprises administering an amount of a 17α-hydroxylase/C_{17,20}-lyase inhibitor, such as compound I, Compound II, or Compound III, and an amount of a glucocorticoid, for example, hydrocortisone, prednisone or dexamethasone. In some embodiments, the method comprises administering about 50 mg/day to about 2000 mg/day of an inhibitor, and about 0.01 mg/day to about 500 mg/day of hydrocortisone. In some embodiments, the method comprises administering about 500 mg/day to about 1500 mg/day of an inhibitor, and about 10 mg/day to about 250 mg/day of hydrocortisone.

In some embodiments, the method for the treatment of a cancer comprises administering an amount of a 17α-hydroxylase/C_{17,20}-lyase inhibitor, including but not limited to Compound I, Compound II, and Compound III, and an amount of a glucocorticoid, such as prednisone. In some embodiments, the method comprises administering about 50 mg/day to about 2000 mg/day of an inhibitor, and about 0.01 mg/day to about 500 mg/day of prednisone. In some embodiments, the method comprises administering about 500 mg/day to about 1500 mg/day of an inhibitor, and about 10 mg/day to about 250 mg/day of prednisone.

In some embodiments, the method for the treatment of a cancer comprises administering an amount of a 17α-hydroxylase/C_{17,20}-lyase inhibitor, such as compound I, Compound II, or Compound III, and an amount of a glucocorticoid, such as dexamethasone. In some embodiments, the method comprises administering about 50 mg/day to about 2000 mg/day of an inhibitor, and about 0.01 mg/day to about 500 mg/day of dexamethasone. In some embodiments, the method comprises administering about 500 mg/day to about 1500 mg/day of an inhibitor, and about 0.5 mg/day to about 25 mg/day of dexamethasone.

Compositions Containing a 17α-hydroxylase/C_{17,20}-lyase Inhibitor and an Additional Therapeutic Agent

In some embodiments, the compositions of the invention comprise a 17α-hydroxylase/C_{17,20}-lyase inhibitor and any one or more of the additional therapeutic agents described herein. Non-limiting examples of forms of administration of the compositions include solutions, suspensions, emulsions, tablets, pills, capsules, powders and sustained-release formulations.

In some embodiments, administration is topical or transdermal administration. In some embodiments, the composition is formulated as a solution, gel, ointment, cream, suspension or salve.

In some embodiments, administration is oral administration. In some embodiments, a composition is a tablet, pill, dragee, troche, capsule, liquid, gel, syrup, slurry, suspension, or emulsion.

In some embodiments, the composition is formulated in a rectal or vaginal composition, such as a suppository or retention enemas comprising conventional suppository bases, such as cocoa butter or other glycerides.

In some embodiments, the composition is a depot preparation. In some embodiments, a depot is administered by implantation (e.g., subcutaneously or intramuscularly) or by intramuscular injection. In some embodiments, the therapeutic agents may are formulated with suitable polymeric or hydrophobic materials (e.g., as an emulsion or suspension in an acceptable oil) or ion exchange resins, or as sparingly-soluble derivatives, for example, as a sparingly-soluble salt.

In some embodiments, the composition is administered using a sustained-release system. In some embodiments, a sustained-release system is a semi-permeable matrix of solid polymers containing the desired composition. Various forms of sustained-release materials have been established and are well known by those skilled in the art. In some embodiments, the sustained-release system is a sustained-release capsule. In some embodiments, the sustained-release capsule releases the composition over a period of hours, days, weeks, or months. In some embodiments, a sustained release capsule releases the composition over a period of at least about 100 days. Depending on the chemical nature and the biological stability of the composition, additional strategies for stabilization may be employed.

In some embodiments, the composition comprises a pharmaceutically-acceptable carrier. The term “carrier” refers to a diluent, adjuvant (e.g., Freund's adjuvant (complete and incomplete)), excipient, or vehicle with which the therapeutic agent(s) is/are administered.

For parenteral administrations, non-limiting examples of carriers include: a sterile diluent, such as water for injection, saline solution, fixed oils, polyethylene glycols, glycerin, propylene glycol or other synthetic solvents; antibacterial agents, such as benzyl alcohol or methyl parabens; antioxidants, such as ascorbic acid or sodium bisulfate; chelating agents, such as ethylenediaminetetraacetic acid; buffers, such as acetates, citrates or phosphates; and agents for the adjustment of tonicity, such as sodium chloride or dextrose. In some embodiments, the parenteral preparation is enclosed in ampules, disposable syringes or multiple dose vials made of glass or plastic.

For oral solid formulations, non-limiting examples of carriers include: fillers, such as sugars, e.g., lactose, sucrose, mannitol and sorbitol; cellulose preparations, such as maize starch, wheat starch, rice starch, potato starch, gelatin, gum tragacanth, methyl cellulose, hydroxypropylmethylcellulose, sodium carboxymethylcellulose, fats and oils; granulating agents; and binding agents, such as microcrystalline cellulose, gum tragacanth or gelatin; disintegrating agents, such as cross-linked polyvinylpyrrolidone, agar, or alginic acid or a salt thereof, such as sodium alginate, Primogel, or corn starch; lubricants, such as magnesium stearate or Sterotes; glidants, such as colloidal silicon dioxide; a sweetening agent, such as sucrose or saccharin; or flavoring agents, such as peppermint, methyl salicylate, or orange flavoring. In some embodiments, the solid dosage form is sugar-coated or enteric-coated using standard techniques.

For intravenous administration, non-limiting examples of carriers include: physiological saline, bacteriostatic water, and phosphate buffered saline (PBS). In some embodiments, the composition is sterile and sufficiently fluid for injection via syringe. In some embodiments, the composition is stable under the conditions of manufacture and storage. In some embodiments, the composition is preserved against contaminating actions of microorganisms, such as bacteria and fungi. In some embodiments, the carrier is a solvent or dispersion medium containing, for example, water, ethanol, polyol (for example, glycerol, propylene glycol, liquid polyethylene glycol, and the like), or a mixture thereof. The proper fluidity may be maintained, for example, by the use of a coating, such as lecithin, by the maintenance of the required particle size in the case of dispersion, and by the use of surfactants. In some embodiments, the action of microorganisms is prevented by various antibacterial and antifungal agents, for example, parabens, chlorobutanol, phenol, ascorbic acid, thimerosal, and the like. In some embodiments, a composition includes isotonic agents, for example, sugars; polyalcohols, such as mannitol or sorbitol; or sodium chloride. In some embodiments, prolonged absorption of the injectable composition is caused by including in the composition an agent which delays absorption, for example, aluminum monostearate or gelatin.

For intravenous administration, the compositions may be formulated in solutions, preferably in physiologically-compatible buffers, such as Hank's solution, Ringer's solution, or physiological saline buffer. In some embodiments, the solution contains formulatory agents, such as suspending, stabilizing and/or dispersing agents. In some embodiments, the compositions are formulated in sterile solutions.

For transmucosal administration, penetrants appropriate to the barrier to be permeated can be used in the formulation. Such penetrants are generally known in the art, and include, for example, for transmucosal administration, detergents, bile salts, and fusidic acid derivatives. In some embodiments, a transmucosal administration agent is a nasal spray or suppository.

For administration by inhalation, the compositions is formulated as an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the dosage unit is determined by providing a valve to deliver a metered amount. Capsules and cartridges of gelatin for use in an inhaler or insufflator are formulated containing a powder mix of the composition and a suitable powder base, such as lactose or starch.

In some embodiments, the pharmaceutical compositions are manufactured by one or more of mixing, dissolving, granulating, dragee-making, levigating, emulsifying, encapsulating, entrapping or lyophilizing processes.

One example of a composition comprising a 17α-hydroxylase/C_{17,20}-lyase inhibitor and an additional therapeutic agent is an oral composition or composition suitable for oral administration comprising an inhibitor in combination with a steroid. In some embodiments, the oral composition is a solid dosage form, such as a pill, a tablet or a capsule. In some embodiments, the amount of the inhibitor is about 10 mg, 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. In some embodiments, the amount of the steroid is about 0.25 mg, 0.5 mg, 0.75 mg, 1.0 mg, 1.25 mg, 1.5 mg, 1.75 mg, 2.0 mg, 2.25 mg, 2.5 mg, 2.75 mg, 3.0 mg, 3.25 mg, 3.5 mg, 3.75 mg, 4.0 mg, 4.25 mg, 4.5 mg, 4.75 mg, 5.0 mg, 7.5 mg, 10 mg, 20 mg, 30 mg, 40 mg or 50 mg. In some embodiments, the steroid is a glucocorticoid.

In one embodiment, the oral composition comprises about 50 mg to about 500 mg of an inhibitor, and about 0.25 mg to about 3.5 mg of a steroid, such as hydrocortisone, prednisone or dexamethasone. In some embodiments, the composition comprises about 50 mg to about 300 mg of an inhibitor, and about 1.0 mg to about 2.5 mg of a steroid, such as hydrocortisone, prednisone or dexamethasone. In another embodiment, the composition comprises about 50 mg to about 300 mg of an inhibitor, and about 0.5 mg to about 3.0 mg of a steroid. In some embodiments, the oral composition is a tablet containing 250 mg of an inhibitor; 1.25 mg or 2.0 mg of a steroid, such as hydrocortisone, prednisone or dexamethasone; and one or more carriers, excipients, diluents and/or additional ingredients. In some embodiments, the oral composition is a capsule containing 250 mg of an inhibitor; 1.25 mg or 2.0 mg of a steroid, such as hydrocortisone, prednisone or dexamethasone; and one or more carriers, excipients, diluents and/or additional ingredients.

The description contained herein is for purposes of illustration and not for purposes of limitation. The methods and compositions described herein can comprise any feature described herein, either alone or in combination with any other feature(s) described herein. Changes and modifications may be made to the embodiments of the description. Also, all references cited above are incorporated herein, in their entirety, for all purposes related to this disclosure.

In some embodiments, the invention provides a method for the treatment of a cancer in a subject, the method comprising administering a therapeutically-effective amount of at least one compound of Formula I:

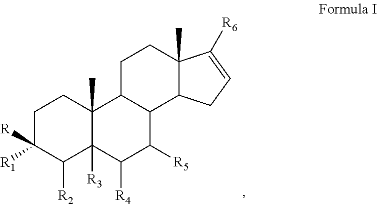

and a therapeutically-effective amount of at least one additional therapeutic agent to a subject having a cancer, wherein:

either R and R₁ are independently H, OH, SH, NH₂, N(R₇), NHR₇, F, OR₇, or O(C═O)R₇; or R and R₁ together form a ketone or an exo-methylene;

each occurrence of R₇ is independently H, C₁-C₈-alkyl, arakyl, alkylaryl, alkoxyalkyl, aryl,

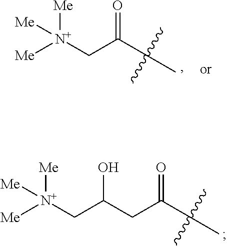

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

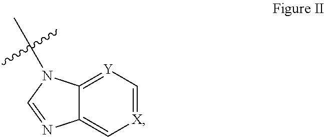

wherein X and Y are independently CH or N, and the bicycle of Formula II is optionally substituted with halogen, chalcogen or C₁-C₄-alkyl,

wherein R₆ is a bicycle of Formula II wherein one of X and Y is N and the other of X and Y is CH when one or both of R and R₁ are

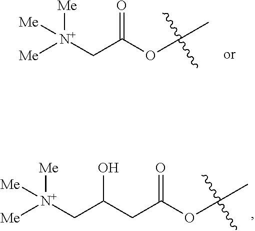

or an analog, a derivative, a metabolite or a pharmaceutically-acceptable salt of any of the foregoing.

In some embodiments, the compound is:

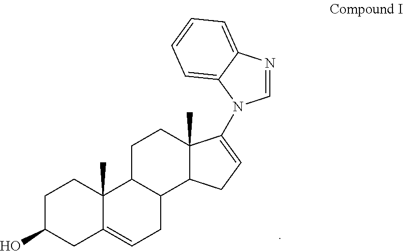

In some embodiments, the compound comprises a sulfonate salt. In some embodiments, the therapeutically-effective amount of the compound is from about 0.01 to about 2000 mg/day. In some embodiments, the additional therapeutic agent is an anti-neoplastic agent, an alkylating agent, an anti-metabolite agent, an antibiotic agent, a hormonal ablation agent, an androgen ablation agent, an anti-androgen agent, or a steroid. In some embodiments, the additional therapeutic agent is mitoxantrone, paclitaxel, docetaxel, leuprolide, goserelin, triptorelin, seocalcitol, bicalutamide, flutamide, hydrocortisone, prednisone or dexamethasone. In some embodiments, the compound and the additional therapeutic agent are administered to the subject in the same composition. In some embodiments, the compound and the additional therapeutic agent are administered separately to the subject. In some embodiments, the cancer is prostate cancer or breast cancer. In some embodiments, the therapeutically-effective amount of the compound is from about 0.01 to about 100 mg/kg/day. In some embodiments, the additional therapeutic agent is mitoxantrone, and wherein the therapeutically-effective amount of mitoxantrone is from about 0.1 to about 20 mg/m². In some embodiments, the additional therapeutic agent is paclitaxel, and wherein the therapeutically-effective amount of paclitaxel is from about 1 to about 175 mg/m². In some embodiments, the additional therapeutic agent is docetaxel, and wherein the therapeutically-effective amount of docetaxel is from about 1 to about 100 mg/m². In some embodiments, the additional therapeutic agent is leuprolide, and wherein the therapeutically-effective amount of leuprolide is from about 0.01 to about 200 mg administered over a period of about 3 days to about 12 months. In some embodiments, the additional therapeutic agent is goserelin, wherein the therapeutically-effective amount of goserelin is about 20 mg administered over a period of about 28 days to about 3 months. In some embodiments, the additional therapeutic agent is triptorelin, wherein the therapeutically-effective amount of triptorelin is from about 0.01 to about 20 mg administered over a period of about 1 month. In some embodiments, the additional therapeutic agent is seocalcitol, and wherein the therapeutically-effective amount of seocalcitol is from about 0.1 to about 500 μg/day. In some embodiments, the additional therapeutic agent is bicalutamide, and wherein the therapeutically-effective amount of bicutamide is from about 1 to about 300 mg/day. In some embodiments, the additional therapeutic agent is flutamide, and wherein the therapeutically-effective amount of flutamide is from about 1 to about 2000 mg/day. In some embodiments, the therapeutically-effective amount of compound I is from about 20 to about 2000 mg/day. In some embodiments, the additional therapeutic agent is hydrocortisone, and wherein the pharmaceutically-effective amount of hydrocortisone is from about 10 to about 250 mg/day. In some embodiments, the additional therapeutic agent is prednisone, and wherein the therapeutically-effective amount of prednisone is from about 5 to about 250 mg/day. In some embodiments, the additional therapeutic agent is dexamethasone, and wherein the therapeutically-effective amount of dexamethasone is from about 0.5 to about 25 mg/day.

In some embodiments, the compound is:

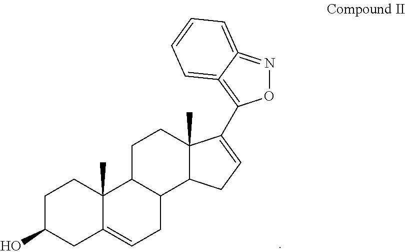

In some embodiments, the compound comprises a sulfonate salt. In some embodiments, the therapeutically-effective amount of the compound is from about 0.01 to about 100 mg/kg/day. In some embodiments, the additional therapeutic agent is mitoxantrone, and wherein the therapeutically-effective amount of mitoxantrone is from about 0.1 to about 20 mg/m². In some embodiments, the additional therapeutic agent is paclitaxel, and wherein the therapeutically-effective amount of paclitaxel is from about 1 to about 175 mg/m². In some embodiments, the additional therapeutic agent is docetaxel, and wherein the therapeutically-effective amount of docetaxel is from about 1 to about 100 mg/m². In some embodiments, the additional therapeutic agent is leuprolide, and wherein the therapeutically-effective amount of leuprolide is from about 0.01 to about 200 mg administered over a period of about 3 days to about 12 months. In some embodiments, the additional therapeutic agent is goserelin, wherein the therapeutically-effective amount of goserelin is about 20 mg administered over a period of about 28 days to about 3 months. In some embodiments, the additional therapeutic agent is triptorelin, wherein the therapeutically-effective amount of triptorelin is from about 0.01 to about 20 mg administered over a period of about 1 month. In some embodiments, the additional therapeutic agent is seocalcitol, and wherein the therapeutically-effective amount of seocalcitol is from about 0.1 to about 500 μg/day. In some embodiments, the additional therapeutic agent is bicalutamide, and wherein the therapeutically-effective amount of bicutamide is from about 1 to about 300 mg/day. In some embodiments, the additional therapeutic agent is flutamide, and wherein the therapeutically-effective amount of flutamide is from about 1 to about 2000 mg/day. In some embodiments, the therapeutically-effective amount of compound I is from about 20 to about 2000 mg/day. In some embodiments, the additional therapeutic agent is hydrocortisone, and wherein the pharmaceutically-effective amount of hydrocortisone is from about 10 to about 250 mg/day. In some embodiments, the additional therapeutic agent is prednisone, and wherein the therapeutically-effective amount of prednisone is from about 5 to about 250 mg/day. In some embodiments, the additional therapeutic agent is dexamethasone, and wherein the therapeutically-effective amount of dexamethasone is from about 0.5 to about 25 mg/day.

In some embodiments, the compound is:

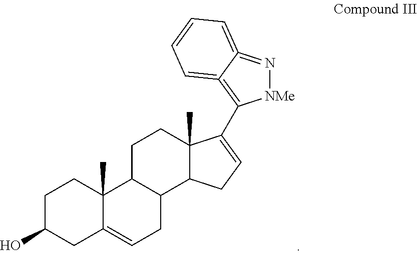

In some embodiments, compound comprises a sulfonate salt. In some embodiments, the therapeutically-effective amount of the compound is from about 0.01 to about 100 mg/kg/day. In some embodiments, the additional therapeutic agent is mitoxantrone, and wherein the therapeutically-effective amount of mitoxantrone is from about 0.1 to about 20 mg/m². In some embodiments, the additional therapeutic agent is paclitaxel, and wherein the therapeutically-effective amount of paclitaxel is from about 1 to about 175 mg/m². In some embodiments, the additional therapeutic agent is docetaxel, and wherein the therapeutically-effective amount of docetaxel is from about 1 to about 100 mg/m². In some embodiments, the additional therapeutic agent is leuprolide, and wherein the therapeutically-effective amount of leuprolide is from about 0.01 to about 200 mg administered over a period of about 3 days to about 12 months. In some embodiments, the additional therapeutic agent is goserelin, wherein the therapeutically-effective amount of goserelin is about 20 mg administered over a period of about 28 days to about 3 months. In some embodiments, the additional therapeutic agent is triptorelin, wherein the therapeutically-effective amount of triptorelin is from about 0.01 to about 20 mg administered over a period of about 1 month. In some embodiments, the additional therapeutic agent is seocalcitol, and wherein the therapeutically-effective amount of seocalcitol is from about 0.1 to about 500 μg/day. In some embodiments, the additional therapeutic agent is bicalutamide, and wherein the therapeutically-effective amount of bicutamide is from about 1 to about 300 mg/day. In some embodiments, the additional therapeutic agent is flutamide, and wherein the therapeutically-effective amount of flutamide is from about 1 to about 2000 mg/day. In some embodiments, the therapeutically-effective amount of compound I is from about 20 to about 2000 mg/day. In some embodiments, the additional therapeutic agent is hydrocortisone, and wherein the pharmaceutically-effective amount of hydrocortisone is from about 10 to about 250 mg/day. In some embodiments, the additional therapeutic agent is prednisone, and wherein the therapeutically-effective amount of prednisone is from about 5 to about 250 mg/day. In some embodiments, the additional therapeutic agent is dexamethasone, and wherein the therapeutically-effective amount of dexamethasone is from about 0.5 to about 25 mg/day.

In some embodiments, the invention provides a method for treating a subject having a refractory prostate or breast cancer, wherein the subject is receiving at least one other treatment for cancer, the method comprising administering a therapeutically-effective amount of at least one 17α-hydroxylase/C_{17,20}-lyase inhibitor in addition to the other treatment the subject is receiving, wherein the 17α-hydroxylase/C_{17,20}-lyase inhibitor is a compound of Formula 1:

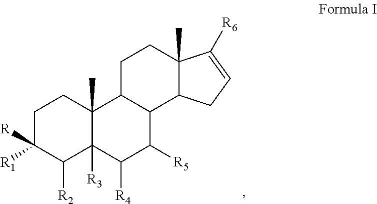

wherein:

either R and R₁ are independently H, OH, SH, NH₂, N(R₇), NHR₇, F, OR₇, or O(C═O)R₇; or R and R₁ together form a ketone or an exo-methylene;

each occurrence of R₇ is independently H, C₁-C₈-alkyl, arakyl, alkylaryl, alkoxyalkyl, aryl,

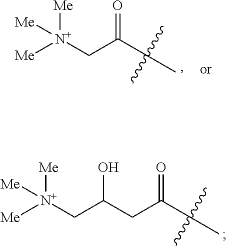

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

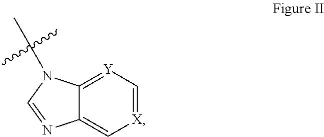

wherein X and Y are independently CH or N, and the bicycle of Formula II is optionally substituted with halogen, chalcogen or C₁-C₄-alkyl,

wherein R₆ is a bicycle of Formula II wherein one of X and Y is N and the other of X and Y is CH when one or both of R and R₁ are

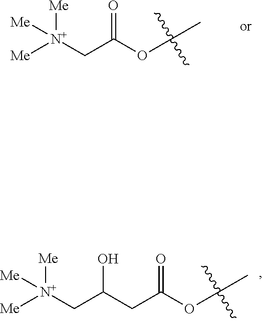

or an analog, a derivative, a metabolite or a pharmaceutically-acceptable salt of any of the foregoing.

In some embodiments, the compound is compound I:

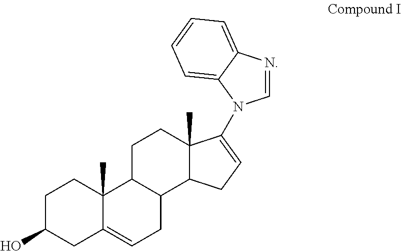

In some embodiments, the compound is a mesylate salt. In some embodiments, the compound is a hydrochloride, bisulfate, or hydrobromide salt.

In some embodiments, the compound is compound II:

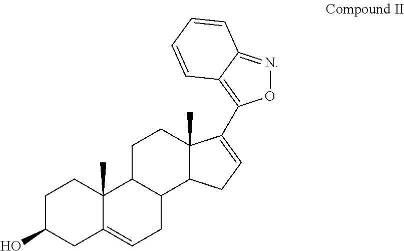

In some embodiments, the compound is a mesylate salt. In some embodiments, the compound is a hydrochloride, bisulfate, or hydrobromide salt.

In some embodiments, the compound is compound III:

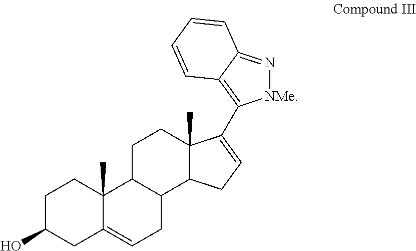

In some embodiments, the compound is a mesylate salt. In some embodiments, the compound is a hydrochloride, bisulfate, or hydrobromide salt. In some embodiments, the therapeutically-effective amount of the compound is from about 20 to about 2000 mg/day. In some embodiments, the other treatment for cancer comprises the administration of an anti-cancer agent, chemotherapy, radiation or surgery.

In some embodiments, the invention provides a pharmaceutical composition for the treatment of a cancer in a subject, the composition comprising a therapeutically-effective amount of at least one 17α-hydroxylase/C_{17,20}-lyase inhibitor, and at least one additional therapeutic agent, wherein the 17α-hydroxylase/C_{17,20}-lyase inhibitor comprises a compound of Formula (1):

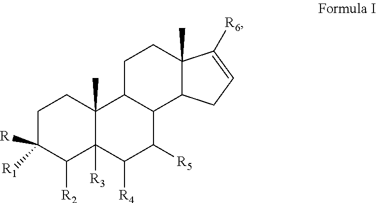

wherein:

R is H or an ester;

R₁ is H, OH, SH, NH₂, N(R₇), NHR₇, F, OR₇, or O(C═O)R₇;

R₇ is independently at each occurrence H, C₁-C₈-alkyl, arakyl, alkylaryl, alkoxyalkyl, or aryl;

R₂, R₃, R₄, and R₅ are independently H, OH, SH, NH₂, or NHR₇, or together with a neighboring R₂, R₃, R₄, or R₅ form an olefinic bond;

R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II:

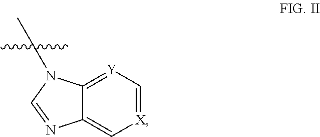

wherein X and Y are independently CH or N, and the bicycle of Formula II is optionally substituted with halogen, chalcogen or C₁-C₄-alkyl,

wherein R₆ is a bicycle of Formula II wherein one of X and Y is N and the other of X and Y is CH when one or both of R and R₁ are

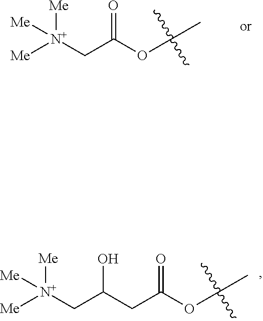

or an analog, a derivative, a metabolite or a pharmaceutically-acceptable salt of any of the foregoing.

In some embodiments, the compound is Compound I:

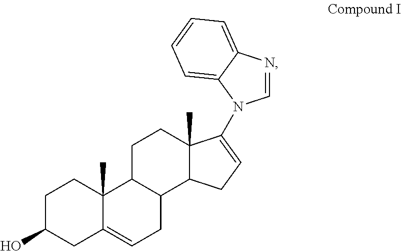

wherein the therapeutically-effective amount of Compound 1 is from about 50 to about 500 mg.

In some embodiments, the compound is Compound II:

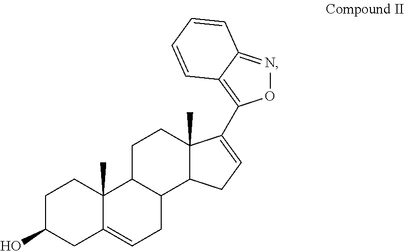

wherein the therapeutically-effective amount of Compound 1 is from about 50 to about 500 mg.

In some embodiments, the compound is Compound III:

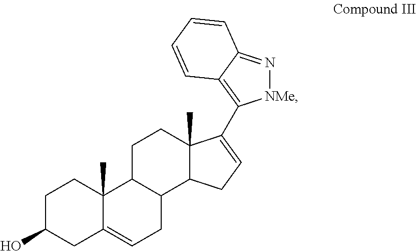

wherein the therapeutically-effective amount of Compound 1 is from about 50 to about 500 mg.

In some embodiments, the additional therapeutic agent is mitoxantrone, paclitaxel, docetaxel, leuprolide, goserelin, triptorelin, seocalcitol, bicalutamide, flutamide, hydrocortisone, prednisone or dexamethasone.

In some embodiments, the invention provides a pharmaceutical composition for the treatment of a cancer in a subject comprising a therapeutically-effective amount of Compound I, Compound II, or Compound III, and a therapeutically-effective amount of a steroid, wherein the composition is suitable for oral administration. In some embodiments, the composition is a solid dosage form. In some embodiments, the composition comprises about 50 to about 500 mg of Compound I, and about 0.25 to about 3.5 mg of the steroid. In some embodiments, the composition comprises about 50 to about 500 mg of Compound II, and about 0.25 to about 3.5 mg of the steroid. In some embodiments, the composition comprises about 50 to about 500 mg of Compound III, and about 0.25 to about 3.5 mg of the steroid. In some embodiments, the steroid is hydrocortisone, prednisone, or dexamethasone. In some embodiments, the composition is a pill, a tablet or a capsule. In some embodiments, the composition is a syrup, emulsion, or suspension.

What is claimed is:

1. A method for the treatment of a cancer in a subject, the method comprising administering a therapeutically-effective amount of at least one compound of Formula I: 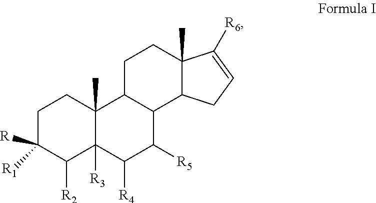
and a therapeutically-effective amount of at least one additional therapeutic agent to a subject having a cancer, wherein:
either R and R₁ are independently H, OH, SH, NH₂, N(R₇), NHR₇, F, OR₇, or O(C═O)R₇; or R and R₁ together form a ketone or an exo-methylene;
each occurrence of R₇ is independently H, C₁-C₈-alkyl, arakyl, alkylaryl, alkoxyalkyl, aryl, 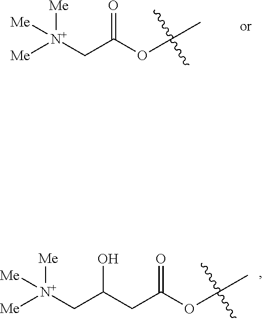
R₂, R₃, R₄, and R₅ are independently H, OH, SH, NH₂, or NHR₇, or together with a neighboring R₂, R₃, R₄, or R₅ form an olefinic bond;
R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, or group, wherein any of the foregoing groups are optionally-substituted, or
a bicyclic structure of Formula II: 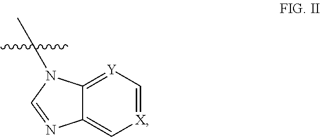
wherein X and Y are independently CH or N, and the bicycle of Formula II is optionally substituted with halogen, chalcogen or C₁-C₄-alkyl,
wherein R₆ is a bicycle of Formula II wherein one of X and Y is N and the other of X and Y is CH when one or both of R and R₁ are 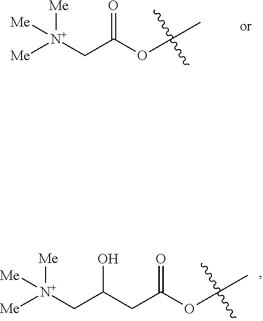
or an analog, a derivative, a metabolite or a pharmaceutically-acceptable salt of any of the foregoing.

2. The method of claim 1, wherein the compound is:

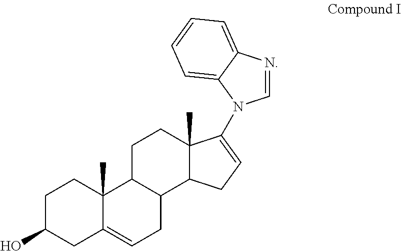

3. The method of claim 2, wherein the compound comprises a sulfonate salt.

4. The method of claim 1, wherein the therapeutically-effective amount of the compound is from about 0.01 to about 2000 mg/day.

5. The method of claim 1, wherein the additional therapeutic agent is an anti-neoplastic agent, an alkylating agent, an anti-metabolite agent, an antibiotic agent, a hormonal ablation agent, an androgen ablation agent, an anti-androgen agent, or a steroid.

6. The method of claim 1, wherein the additional therapeutic agent is mitoxantrone, paclitaxel, docetaxel, leuprolide, goserelin, triptorelin, seocalcitol, bicalutamide, flutamide, hydrocortisone, prednisone or dexamethasone.

7. The method of claim 1, wherein the compound and the additional therapeutic agent are administered to the subject in the same composition.

8. The method of claim 1, wherein the compound and the additional therapeutic agent are administered separately to the subject.

9. The method of claim 1, wherein the cancer is prostate cancer or breast cancer.

10. The method of claim 2, wherein the therapeutically-effective amount of the compound is from about 0.01 to about 100 mg/kg/day.

11. The method of claim 10, wherein the additional therapeutic agent is mitoxantrone, and wherein the therapeutically-effective amount of mitoxantrone is from about 0.1 to about 20 mg/m².

12. The method of claim 10, wherein the additional therapeutic agent is paclitaxel, and wherein the therapeutically-effective amount of paclitaxel is from about 1 to about 175 mg/m².

13. The method of claim 10, wherein the additional therapeutic agent is docetaxel, and wherein the therapeutically-effective amount of docetaxel is from about 1 to about 100 mg/m².

14. The method of claim 10, wherein the additional therapeutic agent is leuprolide, and wherein the therapeutically-effective amount of leuprolide is from about 0.01 to about 200 mg administered over a period of about 3 days to about 12 months.

15. The method of claim 10, wherein the additional therapeutic agent is goserelin, wherein the therapeutically-effective amount of goserelin is about 20 mg administered over a period of about 28 days to about 3 months.

16. The method of claim 10, wherein the additional therapeutic agent is triptorelin, wherein the therapeutically-effective amount of triptorelin is from about 0.01 to about 20 mg administered over a period of about 1 month.

17. The method of claim 10, wherein the additional therapeutic agent is seocalcitol, and wherein the therapeutically-effective amount of seocalcitol is from about 0.1 to about 500 μg/day.

18. The method of claim 10, wherein the additional therapeutic agent is bicalutamide, and wherein the therapeutically-effective amount of bicalutamide is from about 1 to about 300 mg/day.

19. The method of claim 10, wherein the additional therapeutic agent is flutamide, and wherein the therapeutically-effective amount of flutamide is from about 1 to about 2000 mg/day.

20. The method of claim 2, wherein the therapeutically-effective amount of compound I is from about 20 to about 2000 mg/day.

21. The method of claim 20, wherein the additional therapeutic agent is hydrocortisone, and wherein the pharmaceutically-effective amount of hydrocortisone is from about 10 to about 250 mg/day.

22. The method of claim 20, wherein the additional therapeutic agent is prednisone, and wherein the therapeutically-effective amount of prednisone is from about 5 to about 250 mg/day.

23. The method of claim 20, wherein the additional therapeutic agent is dexamethasone, and wherein the therapeutically-effective amount of dexamethasone is from about 0.5 to about 25 mg/day.

24. The method of claim 1, wherein the compound is:

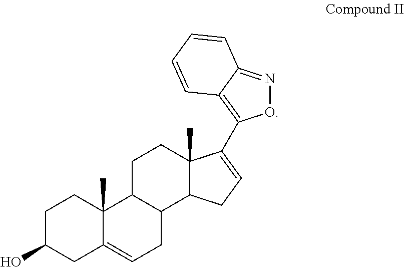

25. The method of claim 24, wherein the compound comprises a sulfonate salt.

26. The method of claim 24, wherein the therapeutically-effective amount of the compound is from about 0.01 to about 100 mg/kg/day.

27. The method of claim 26, wherein the additional therapeutic agent is mitoxantrone, and wherein the therapeutically-effective amount of mitoxantrone is from about 0.1 to about 20 mg/m².

28. The method of claim 26, wherein the additional therapeutic agent is paclitaxel, and wherein the therapeutically-effective amount of paclitaxel is from about 1 to about 175 mg/m².

29. The method of claim 26, wherein the additional therapeutic agent is docetaxel, and wherein the therapeutically-effective amount of docetaxel is from about 1 to about 100 mg/m².

30. The method of claim 26, wherein the additional therapeutic agent is leuprolide, and wherein the therapeutically-effective amount of leuprolide is from about 0.01 to about 200 mg administered over a period of about 3 days to about 12 months.

31. The method of claim 26, wherein the additional therapeutic agent is goserelin, wherein the therapeutically-effective amount of goserelin is about 20 mg administered over a period of about 28 days to about 3 months.

32. The method of claim 26, wherein the additional therapeutic agent is triptorelin, wherein the therapeutically-effective amount of triptorelin is from about 0.01 to about 20 mg administered over a period of about 1 month.

33. The method of claim 26, wherein the additional therapeutic agent is seocalcitol, and wherein the therapeutically-effective amount of seocalcitol is from about 0.1 to about 500 μg/day.

34. The method of claim 26, wherein the additional therapeutic agent is bicalutamide, and wherein the therapeutically-effective amount of bicutamide is from about 1 to about 300 mg/day.

35. The method of claim 26, wherein the additional therapeutic agent is flutamide, and wherein the therapeutically-effective amount of flutamide is from about 1 to about 2000 mg/day.

36. The method of claim 24, wherein the therapeutically-effective amount of compound I is from about 20 to about 2000 mg/day.

37. The method of claim 36, wherein the additional therapeutic agent is hydrocortisone, and wherein the pharmaceutically-effective amount of hydrocortisone is from about 10 to about 250 mg/day.

38. The method of claim 36, wherein the additional therapeutic agent is prednisone, and wherein the therapeutically-effective amount of prednisone is from about 5 to about 250 mg/day.

39. The method of claim 36, wherein the additional therapeutic agent is dexamethasone, and wherein the therapeutically-effective amount of dexamethasone is from about 0.5 to about 25 mg/day.

40. The method of claim 1, wherein the compound is:

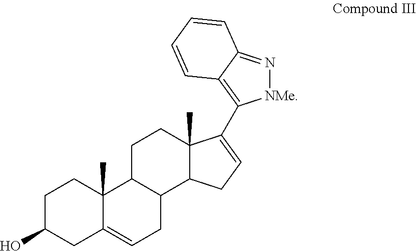

41. The method of claim 40, wherein the compound comprises a sulfonate salt.

42. The method of claim 40, wherein the therapeutically-effective amount of the compound is from about 0.01 to about 100 mg/kg/day.

43. The method of claim 42, wherein the additional therapeutic agent is mitoxantrone, and wherein the therapeutically-effective amount of mitoxantrone is from about 0.1 to about 20 mg/m².

44. The method of claim 42, wherein the additional therapeutic agent is paclitaxel, and wherein the therapeutically-effective amount of paclitaxel is from about 1 to about 175 mg/m².

45. The method of claim 42, wherein the additional therapeutic agent is docetaxel, and wherein the therapeutically-effective amount of docetaxel is from about 1 to about 100 mg/m².

46. The method of claim 42, wherein the additional therapeutic agent is leuprolide, and wherein the therapeutically-effective amount of leuprolide is from about 0.01 to about 200 mg administered over a period of about 3 days to about 12 months.

47. The method of claim 42, wherein the additional therapeutic agent is goserelin, wherein the therapeutically-effective amount of goserelin is about 20 mg administered over a period of about 28 days to about 3 months.

48. The method of claim 42, wherein the additional therapeutic agent is triptorelin, wherein the therapeutically-effective amount of triptorelin is from about 0.01 to about 20 mg administered over a period of about 1 month.

49. The method of claim 42, wherein the additional therapeutic agent is seocalcitol, and wherein the therapeutically-effective amount of seocalcitol is from about 0.1 to about 500 μg/day.

50. The method of claim 42, wherein the additional therapeutic agent is bicalutamide, and wherein the therapeutically-effective amount of bicutamide is from about 1 to about 300 mg/day.

51. The method of claim 42, wherein the additional therapeutic agent is flutamide, and wherein the therapeutically-effective amount of flutamide is from about 1 to about 2000 mg/day.

52. The method of claim 40, wherein the therapeutically-effective amount of compound I is from about 20 to about 2000 mg/day.

53. The method of claim 52, wherein the additional therapeutic agent is hydrocortisone, and wherein the pharmaceutically-effective amount of hydrocortisone is from about 10 to about 250 mg/day.

54. The method of claim 52, wherein the additional therapeutic agent is prednisone, and wherein the therapeutically-effective amount of prednisone is from about 5 to about 250 mg/day.

55. The method of claim 52, wherein the additional therapeutic agent is dexamethasone, and wherein the therapeutically-effective amount of dexamethasone is from about 0.5 to about 25 mg/day.

56. A method for treating a subject having a refractory prostate or breast cancer, wherein the subject is receiving at least one other treatment for cancer, the method comprising administering a therapeutically-effective amount of at least one 17α-hydroxylase/C_{17,20}-lyase inhibitor in addition to the other treatment the subject is receiving, wherein the 17α-hydroxylase/C_{17,20}-lyase inhibitor is a compound of Formula 1: 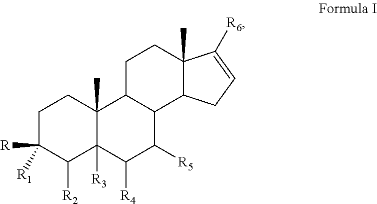
wherein:
either R and R₁ are independently H, OH, SH, NH₂, N(R₇), NHR₇, F, OR₇, or O(C═O)R₇; or R and R₁ together form a ketone or an exo-methylene;
each occurrence of R₇ is independently H, C₁-C₈-alkyl, arakyl, alkylaryl, alkoxyalkyl, aryl, 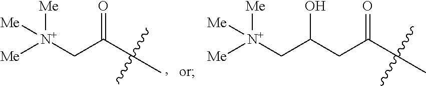
R₂, R₃, R₄, and R₅ are independently H, OH, SH, NH₂, or NHR₇, or together with a neighboring R₂, R₃, R₄, or R₅ form an olefinic bond;
R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II: 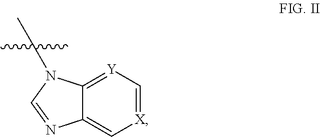
wherein X and Y are independently CH or N, and the bicycle of Formula II is optionally substituted with halogen, chalcogen or C₁-C₄-alkyl,
wherein R₆ is a bicycle of Formula II wherein one of X and Y is N and the other of X and Y is CH when one or both of R and R₁ are 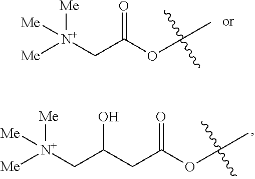
or an analog, a derivative, a metabolite or a pharmaceutically-acceptable salt of any of the foregoing.

57. The method of claim 56, wherein the compound is compound I:

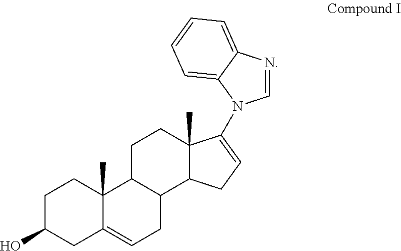

58. The method of claim 57, wherein the compound is a mesylate salt, a hydrochloride salt, a bisulfate salt, or a hydrobromide salt.

59. The method of claim 56, wherein the compound is compound II:

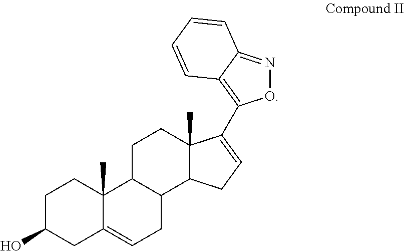

60. The method of claim 59, wherein the compound is a mesylate salt, a hydrochloride salt, a bisulfate salt, or a hydrobromide salt.

61. The method of claim 56, wherein the compound is compound III:

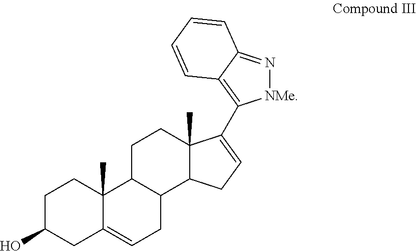

62. The method of claim 61, wherein the compound is a mesylate salt, a hydrochloride salt, a bisulfate salt, or a hydrobromide salt.

63. The method of claim 56, wherein the therapeutically-effective amount of the compound is from about 20 to about 2000 mg/day.

64. The method of claim 56, wherein the other treatment for cancer comprises the administration of an anti-cancer agent, chemotherapy, radiation or surgery.

65. A pharmaceutical composition for the treatment of a cancer in a subject, the composition comprising a therapeutically-effective amount of at least one 17α-hydroxylase/C_{17,20}-lyase inhibitor, and at least one additional therapeutic agent, wherein the 17α-hydroxylase/C_{17,20}-lyase inhibitor comprises a compound of Formula (1): 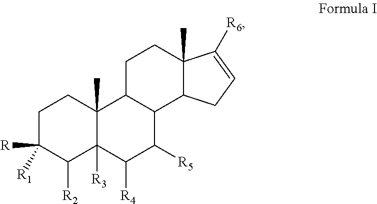
wherein:
R is H or an ester;
R₁ is H, OH, SH, NH₂, N(R₇), NHR₇, F, OR₇, or O(C═O)R₇;
R₇ is independently at each occurrence H, C₁-C₈-alkyl, arakyl, alkylaryl, alkoxyalkyl, or aryl;
R₂, R₃, R₄, and R₅ are independently H, OH, SH, NH₂, or NHR₇, or together with a neighboring R₂, R₃, R₄, or R₅ form an olefinic bond;
R₆ is:
a 1-azaazulen-3-yl; 2-alkylindazol-3-yl; pyrazolo-[1,5-a]-pyridin-3-yl; imidazo-[1,2-a]-pyridin-3-yl; pyrazolo-[2,3-a]-pyrimidin-3-yl; pyrazolo-[2,3-c]-pyrimidin-3-yl; imidazo-[1,2-c]-pyrimidin-3-yl; imidazo-[1,2-a]-pyrimidin-3-yl; 4-alkylpyrazolo-[1,5-a]imidazol-3-yl; 2,1-benzoxazol-3-yl; 2,1-benzthiazol-3-yl; imidazo[2,1-b][1,3]oxazol-5-yl; imidazo[2,1-b][1,3]thiazol-5-yl; imidazo-[2,1-b][1,2]isoxazol-6-yl; or 1,2-benzisoxazol-3-yl, group, wherein any of the foregoing groups are optionally-substituted, or
a bicyclic structure of Formula II: 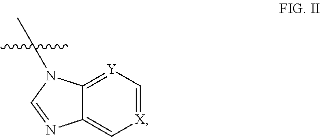
wherein X and Y are independently CH or N, and the bicycle of Formula II is optionally substituted with halogen, chalcogen or C₁-C₄-alkyl,
wherein R₆ is a bicycle of Formula II wherein one of X and Y is N and the other of X and Y is CH when one or both of R and R₁ are 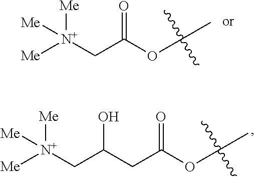
or an analog, a derivative, a metabolite or a pharmaceutically-acceptable salt of any of the foregoing.

66. The composition of claim 65, wherein the compound is Compound I: 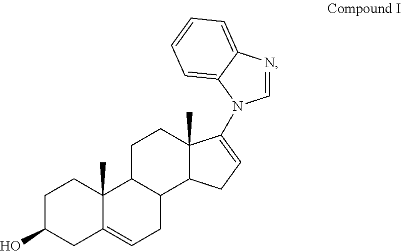
wherein the therapeutically-effective amount of Compound 1 is from about 50 to about 500 mg.

67. The composition of claim 65, wherein the compound is Compound II: 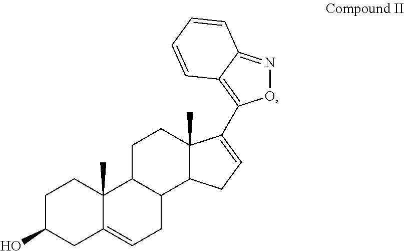
wherein the therapeutically-effective amount of Compound II is from about 50 to about 500 mg.

68. The composition of claim 65, wherein the compound is Compound III: 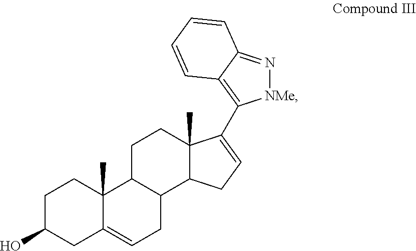
wherein the therapeutically-effective amount of Compound III is from about 50 to about 500 mg.

69. The composition of claim 65, wherein the additional therapeutic agent is mitoxantrone, paclitaxel, docetaxel, leuprolide, goserelin, triptorelin, seocalcitol, bicalutamide, flutamide, hydrocortisone, prednisone or dexamethasone.

70. A pharmaceutical composition for the treatment of a cancer in a subject comprising a therapeutically-effective amount of Compound I, Compound II, or Compound III, and a therapeutically-effective amount of a steroid, wherein the composition is suitable for oral administration.

71. The composition of claim 70, wherein the composition is a solid dosage form.

72. The composition of claim 70, wherein the composition comprises about 50 to about 500 mg of Compound I, and about 0.25 to about 3.5 mg of the steroid.

73. The composition of claim 70, wherein the composition comprises about 50 to about 500 mg of Compound II, and about 0.25 to about 3.5 mg of the steroid.

74. The composition of claim 70, wherein the composition comprises about 50 to about 500 mg of Compound III, and about 0.25 to about 3.5 mg of the steroid.

75. The composition of claim 70, wherein the steroid is hydrocortisone, prednisone, or dexamethasone.

76. The composition of claim 71, wherein the composition is a pill, a tablet or a capsule.

77. The composition of claim 70, wherein the composition is a syrup, emulsion, or suspension.
